(19) United States
(12) Patent Application Publication
(10) Pub. No.: US 2014/0238588 A1
(43) Pub. Date: Aug. 28, 2014

(54) DEVICES AND METHODS FOR INTRODUCING A DISPOSAL BAG INTO THE PRODUCTION OF DISPOSABLE DIAPERS

(71) Applicant: SAMUEL KARSENTI, Jerusalem (IL)

(72) Inventor: SAMUEL KARSENTI, Jerusalem (IL)

(21) Appl. No.: 13/779,777

(22) Filed: Feb. 28, 2013

(51) Int. Cl. A61F 13/00

(52) U.S. Cl. CPC A61F 13/00987
USPC 156/227; 156/510

(57) ABSTRACT

The invention discloses methods and devices for inserting compressed plastic disposal bags into a diaper production line. The bags are generally placed between the inner rear wall and an absorbent layer of the diaper so as to allow both facile access to the bag as well as protection of the bag from human waste. A hole cut in the rear wall allows for exposure of a tab adapted to be pulled to deploy the entire disposal bag. The invention, in some embodiments, may be integrated into diaper production machines.

### FIELD AND BACKGROUND OF THE INVENTION

The present invention, in some embodiments thereof, relates to methods and devices for producing disposable diapers, pants, tampons and the like, wherein individual disposal bags are included in the disposable products. The instant invention, in some embodiments, describes systems and methods for preparing a disposal bag, transferring said bag to a diaper production machine and allowing for final production of disposable diapers with inserted disposal bags.

Over the last four decades, many improvements have been made in the development of disposable diapers. The diapers' appearance, absorbency, fit, softness, size and shaping have all been improved in many different ways.

However, diaper disposal still remains a problem with respect to hygiene, smell and space. Some efforts towards improving diaper disposal appear in the patent literature.

U.S. Pat. No. 4,034,760 discloses a combined disposable diaper and disposal bag wherein a thin plastic sheet is affixed over the waterproof side of a conventional disposable diaper and sealed to it along a portion of its periphery to form a permanently attached waterproof pocket with part of the sheet that can be turned inside out to form a bag for the used disposable diaper and to form a flap with the remainder of the sheet which can be closed over the bag opening to seal the bag.

U.S. Pat. No. 4,604,096 discloses a diaper assembly with integral disposal system, including a disposable diaper and an envelope integrally carried on the outside of the diaper, that has a mouth for receiving the diaper when soiled. One or more elements engage a section of the diaper with another section of the diaper to secure it on a wearer in a use configuration and restrict the mouth of the envelope in a disposal configuration.

U.S. Pat. No. 4,706,845 describes a unit combining a diaper dispenser and disposal drawer. The unit consists of two sections, an upper dispensing section and a lower disposal section, which provide a single unit for new and soiled diapers, as well as other supplies used in changing a baby. The upper dispensing section has a number of dividers which define vertical shafts. Diapers are placed in these shafts and are removable at the bottom thereof through adjustable slots. The slots of the upper dispensing section are adjustable by using a removable shelf. The vertical shafts are also adjustable in cross-sectional size to accommodate different sized diapers. A slidable front panel permits easy access to one or more of the vertical shafts for reloading. The bottom disposal section comprises a cabinet generally rectangular in shape with a pivoting, top-opening door. This disposal section may contain a separate bin, such as a plastic bag or bucket which is easily removed to dispose of soiled diapers and other waste. The door additionally has a liner along its edges making the lower section of the unit airtight and thus odor-free. Space is also provided to allow the user to place a deodorizer within the lower section.

French Patent Application No. FR2699401A1 describes a nappy (diaper) having a pouch built into its outer surface, containing a plastic bag which can be withdrawn through a hole in the outer wall of the pouch by pulling on a projecting tab. Once the bag has been withdrawn it can be turned inside out, enclosing the used nappy, pulling its top closed with a draw-string. The nappy can then be disposed of hygienically.

US Patent Application No. 2002029546 discloses a method for disposing of a soiled disposable diaper comprising the steps of: providing a bag made of a plastic material, the bag having a front panel and back panel integrally sealed together along the left and right side edges and the bottom side edge thereof and having an opening at a top edge of one of the panels, a seal flap connected to and being integral with a top edge of the other one of the panels, a strip of adhesive extending across the sealing flap and a cover strip extending across the front of the seal flap over the strip of adhesive, removing the cover strip, placing a soiled diaper in the plastic bag, folding the seal flap over the opening in the bag and onto an adjacent panel, pressing the strip of adhesive against the adjacent panel to generally seal the bag, and placing the generally sealed bag in a waste disposal container.

US Patent Application No. 2004176735 describes a packaged diaper, including a diaper, having a first, a nominal size, and a second, reduced size, the reduced size of the diaper being convenient for carriage and storage of the diaper. The diaper is disposed in an encasement in the second, reduced size. The encasement confines the diaper so that the diaper is retained in its reduced size by the encasement and so that the diaper is returned to its nominal size upon opening of the encasement.

European Patent Application No. EP0888766A describes a disposable nappy having a body, an outer surface of the body, and a pocket formed on the outer surface of the nappy for containing the body of the nappy when the used nappy is folded and the pocket is inverted neatly and easily, prior to disposal.

British Patent Application No. GB2414676A describes a disposable nappy which has a nappy sack incorporated into the back of the nappy. Preferably the nappy sack is released by pulling a tab which also forms the handle of the nappy sack. The nappy sack may envelop the nappy and be tied for hygienic disposal. This invention may eliminate the need for using separate nappy sacks.

French Patent Application No. FR2898488A describes an integrated device for wrapping used and rolled disposable nappy by a circular stretchable elastic fibrous membrane, which is integrated in the nappy in its external back and upper surface. The membrane comprises an orifice at the center of the membrane, which is provided with an elastic ring, and stretches when user grabs a central elastic ring.

International Patent Application WO/2000/016679A describes a system for disposing of products of metabolism, especially human products of metabolism. The system consists of a base part which can be reused and a disposable multi-layer liner which is essentially adapted to fit the base part and is detachably fixed to the same. The invention also relates to a multi-layered, bag-shaped liner and to the use of this system and the liner in the fields of nursing, care of the elderly and care of infants.

German Patent Application No. DE102005007004A, to Gandolfo, describes a disposable nappy, which has a disposal bag stored between the inner absorbent layer and the impermeable outer layer. The disposal bag is permanently connected to the outer layer and can be pulled out from the inside of the nappy, via a slit-type opening to permit its disposal after use.

US Patent Application No. 2006282056, to Ramsey, describes one or more improvements includes a fluid-impermeable bag attached to or integral with a disposable diaper, the bag having closure elements on the outside of the front and back panels of the uninverted bag. After use, the bag is inverted over the soiled diaper and the closure elements interact, thereby closing and sealing the soiled diaper inside the bag. In some embodiments the bag is covered by a removable fabric element, such as a gauze panel, which may be decorated.

International Patent Application WO2010/103516 to Karsenti teaches a diaper assembly comprising an integral disposal system, the diaper assembly comprising a disposable diaper comprising an outer layer comprising an aperture on an outer surface thereof, an inner layer and an absorbent layer disposed between said outer layer and said inner layer and a bag disposed between said outer layer and said inner layer, said bag being folded and adapted to be retrieved via the aperture and to receive and enclose said diaper, wherein the absorbent layer is of a greater thickness than the height of the folded bag.

International Patent Application WO2008/023363 describes a bag attachable to an article to permit convenient disposal of the article after use, comprising a bottom section having an adherent outer surface for attachment to the article, a top section overlying the bottom section, and a tubular main section joined at one end to the bottom section, the tubular main section being of an expansible construction, normally in a contracted condition between the top and bottom sections, but being expandable to an expanded condition to define a compartment for receiving the article after use, at least one of the sections being formed with an opening to be used when introducing the used article into the compartment formed by the tubular main section in its expanded condition.

Despite the teachings of the publications presented hereinabove, there are no products available on the market that address all of the problems of diaper disposal.

Moreover, the inventions described hereinabove do not describe or reasonably suggest the methods by which such diapers are actually produced.

### SUMMARY OF THE INVENTION

It is therefore a purpose of the present invention, in some embodiments, to provide methods and devices for producing diapers with a folded or compressed disposal bag tucked between an outer plastic portion of the diaper and an absorbent inner web layer.

The invention includes a method for creating a disposable diaper with an internal bag for diaper disposal, including the following: providing a plurality of connected plastic bags; cutting the roll into individual bags; folding each of the individual bags into a compressed shape; gluing a tab to a first side of the plastic bag, wherein the tab is adapted to be pulled for bag deployment when needed; forming a roll of compressed bags, wherein consecutive compressed bags are transiently attached one to another; providing an outer plastic layer corresponding to the back of the disposable diaper; cutting a hole in the outer plastic layer to allow for external exposure of the tab; transferring the roll of compressed bag to a diaper production machine, wherein a second side of the bag is glued to the outer plastic layer corresponding to the back of the disposable diaper and the tab is exposed through the hole; covering the plastic bag with an absorbent web material, wherein the plastic bag sits between the outer plastic layer and the absorbent web material; and, completing production of the disposable diaper, wherein the plastic bag rests in a back portion of the disposable diaper and is adapted to be opened for receiving the diaper when soiled.

In one embodiment of the method, the step of forming is performed by placing each compressed bag into a pair of circular holders, wherein the holders are placed at the outer edges of each of the compressed bag and are glued with circular holders from a neighboring compressed bag to form a roll.

In another embodiment of the method, the step of forming is performed by placing the compressed bag into a larger holding bag having a hole to allow the tab to protrude, and wherein consecutive holding bags are glued together to form a roll.

In another embodiment of the method, the compressed bag is glued on a first side to an inner side of the larger holding bag.

In another embodiment of the method, the plastic bags are biodegradable.

In another embodiment of the method, the diaper is used for a toddler, child, medically unwell individual, or adult.

In another embodiment of the method, the diaper is realized as a woman's tampon.

In another embodiment of the method, the compressed shape has an exposed surface area of approximately 10% of that of the surface area of an uncompressed individual bag.

In another embodiment of the method, the gluing is performed by any of the following means: spray, direct application, contact application, heating, or by hand.

In another embodiment of the method, the outer plastic layer is realized as a long sheet of plastic adapted to be formed into an outer portion of the diaper.

In another embodiment of the method, the cutting is performed with a knife, laser, cutting tool, or razor.

In another embodiment of the method, the absorbent web material is adapted to absorb extruded bodily fluids.

In another embodiment of the method, the circular holders are made of plastic, paper, rubber, or polymer material.

The invention also includes a method for positioning disposable bags during production of a plurality of disposable diapers, including the following: providing a plurality of plastic bags; folding each of the individual bags into a compressed shape; gluing a tab to a first side of each plastic bag, wherein the tab is adapted to be pulled for bag deployment when needed; forming a roll of compressed bags, wherein consecutive compressed bags are transiently attached one to another; providing an outer plastic layer corresponding to the back of the disposable diapers; cutting a plurality of holes in the outer plastic layer to allow for external exposure of the tab; transferring the roll of compressed bags to a diaper production machine, wherein a second side of each of the bags is glued to the outer plastic layer corresponding to the back of the disposable diapers and the tabs are exposed through the holes; and, covering the plastic bags with an absorbent web material, wherein the plastic bags sit between the outer plastic layer and the absorbent web material.

In one aspect of the method, there is a further step of completing production of the disposable diapers, wherein the plastic bags rest in a back portion of the disposable diapers and is adapted to be opened for receiving the diapers when soiled.

In another aspect of the method, the compressed bags in the roll are transiently attached directly or indirectly one to another in the roll.

In another aspect of the method, the diaper production machine is adapted to produce hundreds of diapers or more per minute.

In another aspect of the method, the tabs are made from plastic, paper, or a polymer material.

In another aspect of the method, the step of transferring occurs in proximity to the diaper production machine.

In another aspect of the method, the step of completing includes forming the disposable diapers, cutting individual diapers, arranging the disposable diapers in groups of diapers and packaging the groups of diapers.

In another aspect of the method, the tab is adapted to be pulled without a user soiling his or her hands.

The invention additionally includes device for inserting a plastic disposal bag during disposable diaper production, including: a diaper production machine adapted to produce hundreds or more disposable diapers per minute; a roll of compressed plastic bags, wherein consecutive compressed plastic bags are connected one to another, and wherein each compressed plastic bag has associated with it a pull tab glued to a first side of the compressed plastic bag; a cutting unit adapted to cut the plastic bags into individual bags for delivery to the diaper production machine; a roll of plastic sheet adapted to be fed through the diaper production machine; a cutting element adapted to form regularly-spaced cuts in the plastic sheet; a gluing element adapted to glue the individual bags onto the plastic sheet in general proximity to the cuts, wherein the tabs are exposed externally through the cuts in the plastic sheet; and, a roll of absorbent web material adapted to be placed over the compressed plastic bags associated with the plastic sheet.

In one aspect of the device, there is a plurality of tabs, wherein the tabs are adapted to be attached to the individual plastic bags and allow for deployment of the bags.

In another aspect of the device, the compressed plastic bags are connected one to another through the agency of a larger holding bag.

In another aspect of the device, the cutting tool is associated with the diaper production machine.

In another aspect of the device, the glue application element includes a spray component.

In another aspect of the device, the diaper production machine is selected from one of the following vendors: Joa, Zuiko, or Fameccanica.

Unless otherwise defined, all technical and/or scientific terms used herein may have the same general meanings as commonly understood by a practitioner of ordinary skill in the art to which the invention pertains. Diapers herewith described are generally disposable. They may be for babies, children, the physically ill or adults. The term “diapers” may additionally include tampons, baby and children pants and the like. “Absorbent web material” or “absorbent pad” may generally refer to absorbent material placed on the inside of diapers to absorb urine and other materials released from the body.

Such material is generally described herewith: http://www.madehow.com/Volume-3/Disposable-Diaper.html. The plastic bags employed in the instant invention may generally be made from a recyclable plastic and are generally biodegradable. “Plastic” may refer to any natural or unnatural material having an organic property, including but not limited to polymers, synthetic materials, and composite materials. “Compressed” with respect to a plastic disposal bag may generally refer to a plastic bag that is folded on itself in full or in part or otherwise made to have a smaller physical footprint than the original bag itself has. Non-limiting examples of diaper production machines include but are not limited to Curt G. Joa J-4 machine (http://www.joa.com/products/products_j4bdm.html), the Joa J-6 machine (http://www.joa.com/products/products_j6bdm.html), Fameccanica FA-X P10 (http://www.fameccanica.com/index.php?option=com_content&view=article&id=301&Itemid=203&lang=en), Fameccanica FIX P5 (http://www.fameccanica.com/index.php/?option=com_content&view=article&Itemid=73&task=findkey&keyref=adult), as well as Zuiko diaper machines for children, adults and baby pants (http://www.zuiko.co.jp/english/01/01_diaper.html).

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. It is noted that similar elements in various drawings will have the same number, advanced by the appropriate multiple of 100.

In the drawings:

FIG. 1 shows a method associated with the instant invention;

FIGS. 2A-2E show schematic views related to an embodiment of the instant invention;

FIG. 3 shows a method embodiment associated with the instant invention;

FIG. 4 shows a schematic view of an unfolded a folded disposable plastic bag; and,

FIG. 5 shows a schematic view of an embodiment of the instant invention associated with an Example herein provided.

### DESCRIPTION OF SPECIFIC EMBODIMENTS OF THE INVENTION

The present invention, in some embodiments thereof, relates to systems and devices for allowing addition of a disposal bag to a disposable diaper. Without being bound by any particular theory, the following discussion is offered to facilitate understanding of the invention. The present invention, in some embodiments, provides for facile production of diapers with a disposal bag included within each diaper.

As described above, there are inventions relating to the inclusion of a disposal bag with disposable diapers. Such bags offer convenience for disposal of a soiled diaper or the like, without concern for smell or body wastes being exposed to others. While the prior art describes bags and their relationships to diapers, the prior art does not discuss or reasonably suggest how to insert disposal bags into disposable diapers that are produced at the rate of hundreds to thousands of units per minute.

For purposes of better understanding, some embodiments of the present invention are illustrated in the figures of the drawings.

### First Embodiment

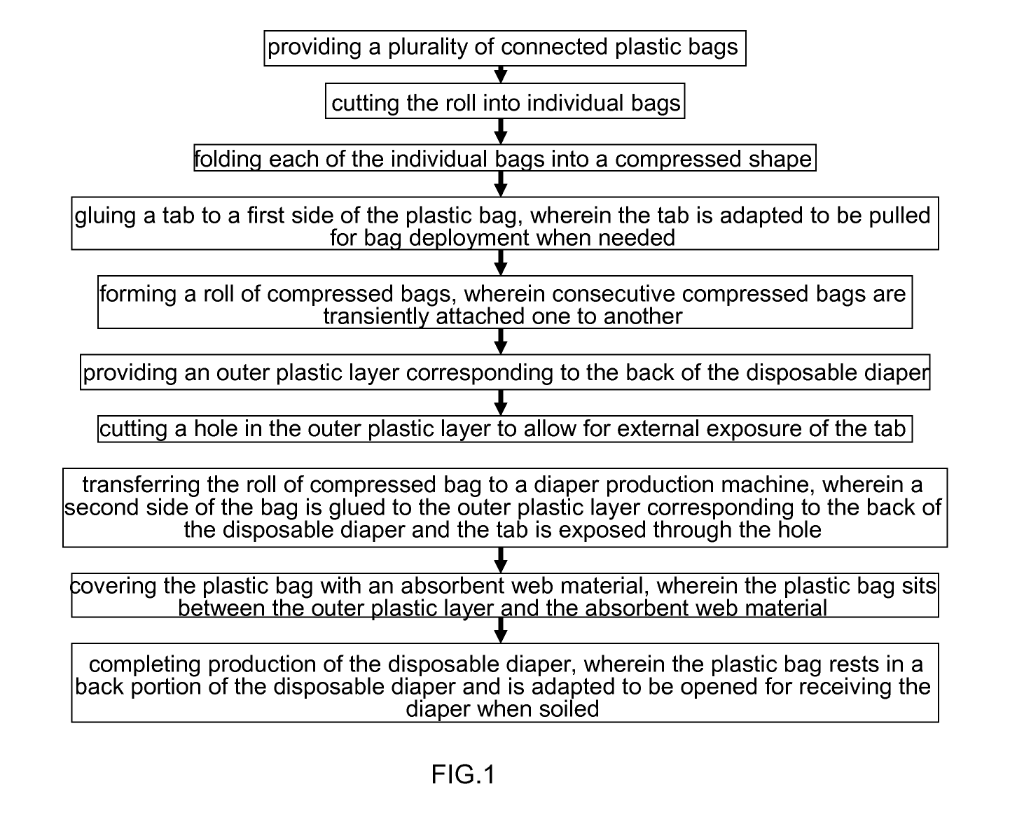

Attention is turned to FIG. 1 which shows an embodiment of the instant invention. The method includes the following steps: providing a plurality of connected plastic bags; cutting the roll into individual bags; folding each of the individual bags into a compressed shape; gluing a tab to a first side of the plastic bag, wherein the tab is adapted to be pulled for bag deployment when needed; forming a roll of compressed bags, wherein consecutive compressed bags are transiently attached one to another; providing an outer plastic layer corresponding to the back of the disposable diaper; cutting a hole in the outer plastic layer to allow for external exposure of the tab; transferring the roll of compressed bag to a diaper production machine, wherein a second side of the bag is glued to the outer plastic layer corresponding to the back of the disposable diaper and the tab is exposed through the hole; covering the plastic bag with an absorbent web material, wherein the plastic bag sits between the outer plastic layer and the absorbent web material; and, completing production of the disposable diaper, wherein the plastic bag rests in a back portion of the disposable diaper and is adapted to be opened for receiving the diaper when soiled. In this embodiment, plastic disposal bags are connected as provided, though free plastic bags are also amenable for use in the instant invention. The bags are first cut into individual bags and then folded so as to compress their overall footprint. A disposal bag must obviously be larger than the diaper it comes to dispose; placing such a bag “as is” into a diaper would be impractical due to the former's larger dimensions; thus, plastic bags are first compressed or folded by any relevant means to make them smaller in overall dimensions (and thus thicker). A typical bag may be folded over itself to reduce its apparent length and width by factors of five to ten.

In one embodiment of the method, the step of forming is performed by placing each compressed bag into a pair of circular holders, wherein the holders are placed at the outer edges of each of the compressed bag and are glued with circular holders from a neighboring compressed bag to form a roll. In another embodiment, the step of forming is performed by placing the compressed bag into a larger holding bag having a hole to allow the tab to protrude, and wherein consecutive holding bags are glued together to form a roll. In another embodiment, the compressed bag is glued on a first side to an inner side of the larger holding bag. In another embodiment of the method, the plastic bags are biodegradable. In another embodiment, the diaper is used for a toddler, child, medically unwell individual, or adult. In another embodiment of the method, the diaper is realized as a woman's tampon. In another embodiment, the compressed shape has an exposed surface area of approximately 10% of that of the surface area of an uncompressed individual bag. In another embodiment of the method, the gluing is performed by any of the following means: spray, direct application, contact application, heating, or by hand. In another embodiment of the method, the outer plastic layer is realized as a long sheet of plastic adapted to be formed into an outer portion of the diaper. In another embodiment of the method, the cutting is performed with a knife, laser, cutting tool, or razor. In another embodiment of the method, the absorbent web material is adapted to absorb extruded bodily fluids. In another embodiment of the method, the circular holders are made of plastic, paper, rubber, or polymer material.

After the disposal bags have been cut and compressed in size, they are joined together to form a roll. As is well-known in the art, virtually all components—plastics, webbing, etc.—are introduced in the form of rolls to a diaper manufacturing machine. Diaper production rates are generally quoted in units produced per minute or in meters of (roll) material used per minute. Tabs may be added to the compressed bags; in some embodiments, tabs are not required and the bag itself may partly stick out the back for easy deployment. The compressed bags may be joined together by any means. Heat joining, gluing, attaching, holding together are some means by which the compressed bags may be held consecutively one to another to form a roll. The roll of compressed plastic bags including tabs is then ready to be introduced to the diaper production line. A plastic sheet spooled out of a roll serves as the backing for diapers. Typically, this backing is uncut; in the instant invention, a cut is introduced in the plastic sheet at predetermined intervals, wherein said intervals correspond more or less to diaper length. Thus, a cut in the plastic will be followed by another cut in the plastic 20-30 centimeters later on the roll. A compressed plastic bag is placed in proximity to the cut, glued in place and the tab is arranged to jut out from the hole in the plastic sheet. Thus, the compressed plastic bag sits glued on a first side of the plastic sheet, while its associated tab juts out of the hole to appear on a second side of the plastic sheet. The bag and the plastic sheet are then covered in an absorbent web material which serves to absorb urine and the like. At this point, the glued bag sits between the back of the diaper and absorbent material facing the back of the diaper. The tab sticks out from the back of the diaper. It is obvious that the same arrangement could be made with a front region of the diaper, but then a child or toddler might pull the tab to unnecessarily deploy the disposal bag.

Diaper production continues with addition and modification of materials as required. The long roll of diaper material is cut into individual diapers which are arranged and packaged prior to shipping. The instant invention does not interfere or necessarily modify any steps of diaper production after insertion of the compressed disposal bag between the back plastic and absorbent webbing material of the diaper.

### Second Embodiment

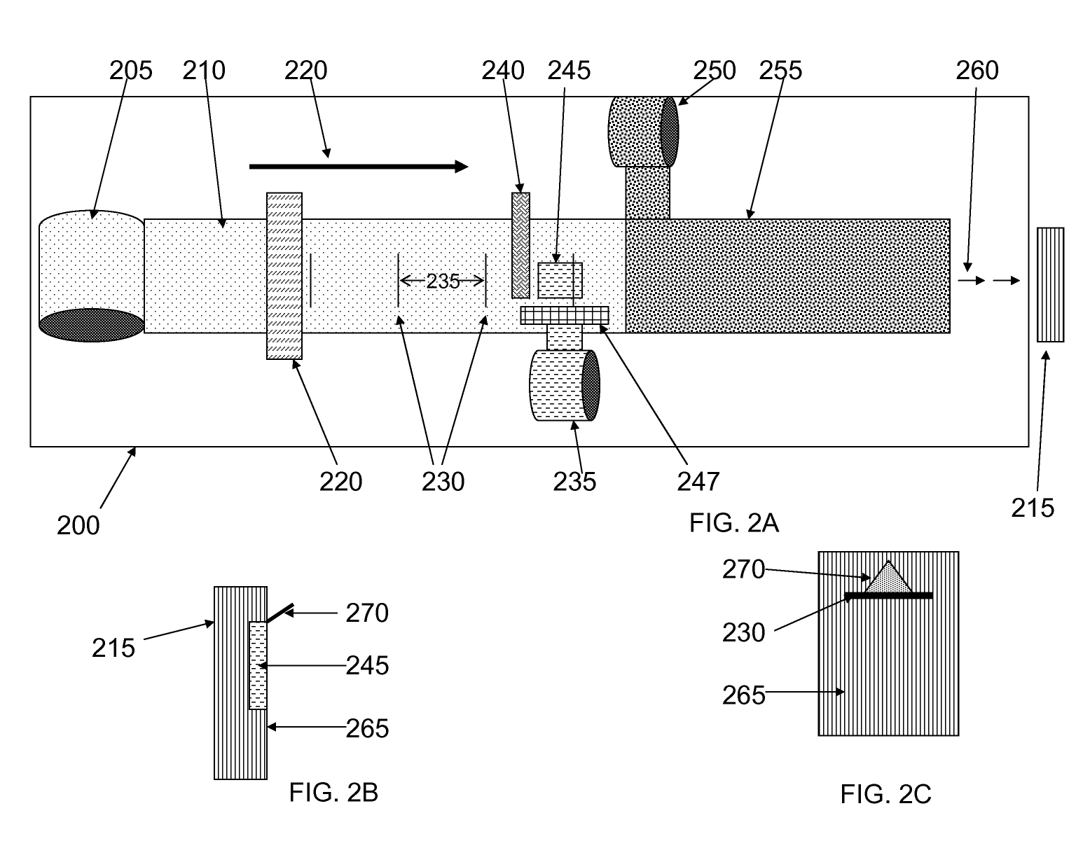

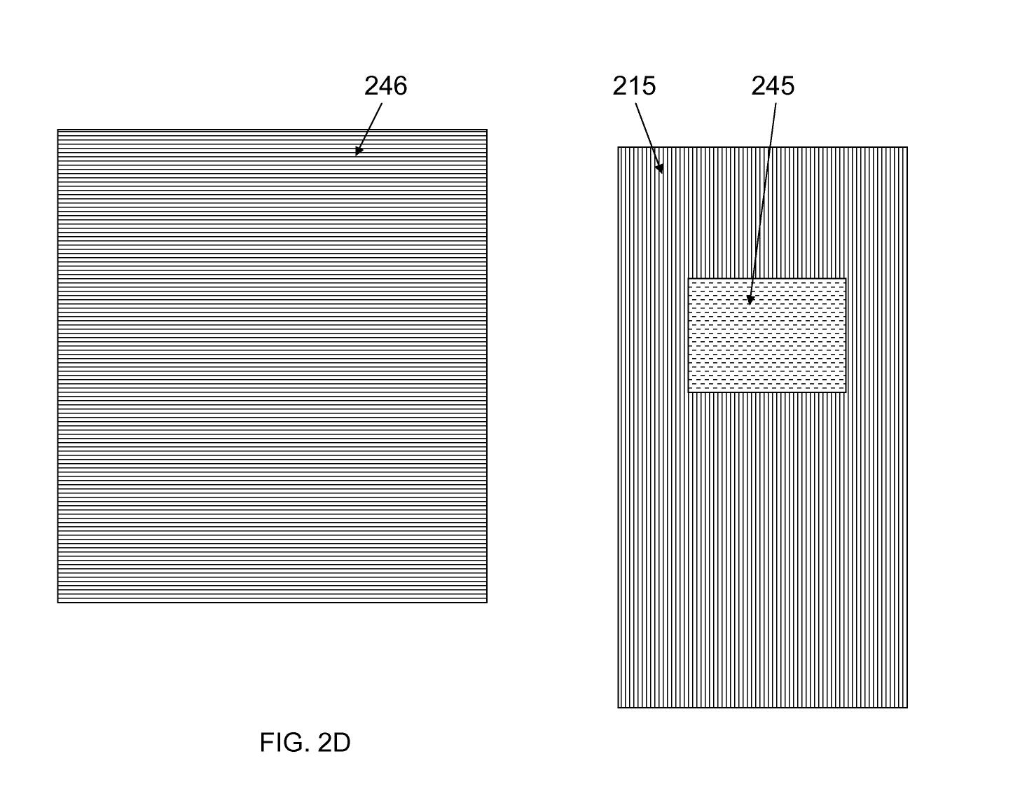

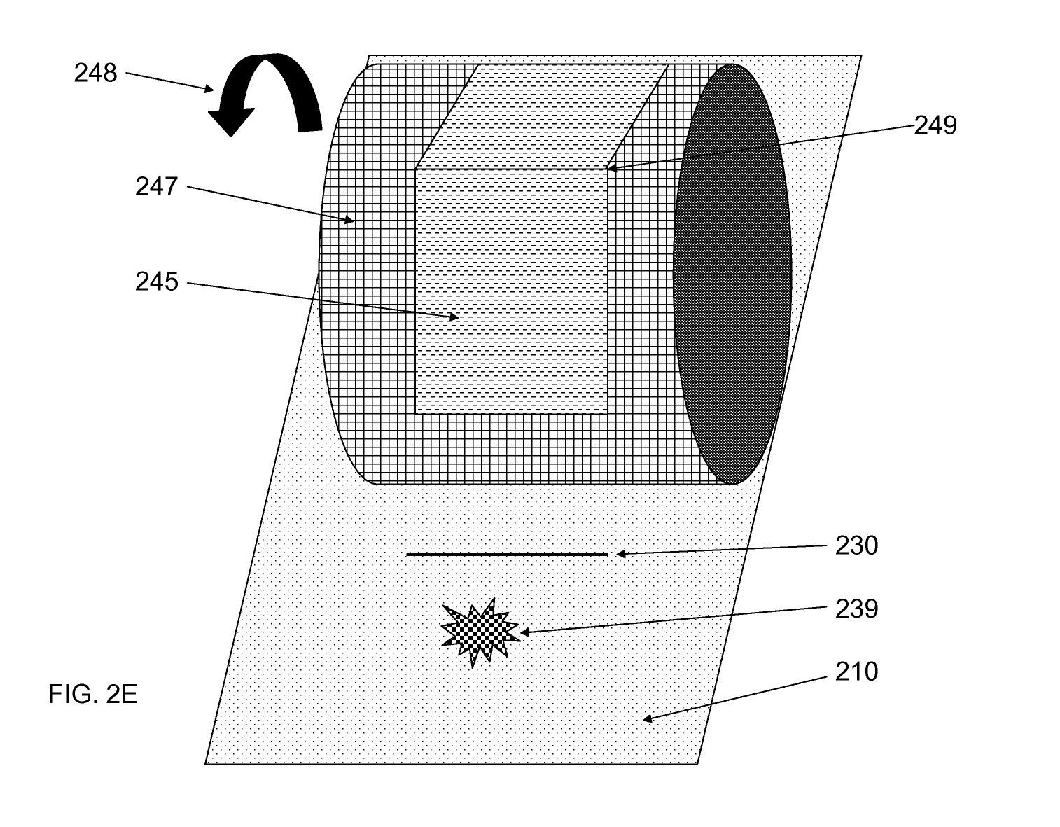

Attention is turned to FIG. 2A which shows a schematic drawing of an embodiment according to the instant invention. A modified diaper production machine 200 includes a roll 205 of plastic sheet 210 that serves as the outer surface for a finished diaper 215. The plastic sheet 210 is directed 220 at speeds of tens of meters per minute towards a cutting element 225 that places cuts 230 in said plastic sheet 210 in a direction perpendicular to the direction 220 of motion of said sheet 210. The cutting element 225 places a new cut 230 at a predetermined spacing 235 along the sheet. The cuts 230 may be in the same direction as or perpendicular to the direction of movement of the plastic sheet 210. A gluing element 240 applies glue on the plastic sheet 210 to the left of each cut 230. A roll 235 of folded plastic disposal bags 245 is provided as is a cutting unit 247 which cuts off individual plastic bags 245 which are then glued to the plastic sheet 210 in the general proximity of a cut 230. The cutting unit 247 may also include elements to press the cut disposal bag 245 in place. The cutting unit may alternatively be in the form of a roller that receives uncut bags 245 from the roll 235 and then cuts and pastes disposal bags 245 in place on the plastic sheet 210. A roll 250 of absorbent web material 255 is provided and the web material 255 is placed over the plastic sheet 210 and its associated glued plastic disposal bags 245 and cuts 230 (no longer seen under the absorbent web material 255). Additional equipment and materials 260 are employed to finish preparation of individual finished diapers 215. A typical roll 205, 235 & 255 may include hundreds of meters of the appropriate material used in diaper preparation. It is understood that additional layers of material above or below the plastic bags 245 may be included in diaper production and are not shown for the purposes of clarity. As shown in the instant embodiment, the disposable plastic bag 245 is positioned on the inside of the plastic sheet that forms the outer most layer of a finished diaper 215.

Attention is turned to FIG. 2B which shows a schematic cut-away view of a finished diaper 215 that includes a folded plastic disposal bag 245 glued to an inner side of the plastic rear 265 section of the diaper 215. A tab 270 associated with the plastic disposal bag 245 is shown jutting out from the rear 265 section of the diaper 215. Pulling the tab 270 leads to deployment of the plastic disposal bag 245 which is used to contain the diaper 215, after the latter becomes soiled (not shown). The tab 270 extends from the cut 230 on the rear 265 portion of the diaper 215 as shown in FIG. 2C.

The cutting element 225 and the cutting unit 247 may use any means of cutting including but not limited to laser, knife, scissors, razor, and press. The gluing element 240 may apply any form of glue or adhesive by any means including but not limited to spray, liquid or solid application, heat treatment, pressure, or melting.

Attention is turned to FIG. 2D which shows an unfolded plastic disposal bag 246 on the left and the folded/compressed plastic disposal bag 245 associated with a finished diaper 215 on the right. The unfolded plastic bags 246 are generally compressed approximately 90% in exposed surface area so that the folded plastic bag 245 will fit easily in the diaper 215.

Attention is turned to FIG. 2E which shows one version of a cutting unit 247 realized as a drum adapted to accept connected plastic bags 245. The drum turns 248 so as to place a plastic disposal bag 245 in proximity to a cut 230 in the plastic sheet 210 in the general vicinity of glue 239 placed on the plastic sheet 210, as shown. The cutting unit drum 247 turns 248 and deposits the plastic bag 245, which is cut along a connection zone 249 and the drum presses the separated bag 245 into place over the glue 239. The cutting unit 247 as drum thus may serve to both cut folded plastic bags 245 and place them firmly over the plastic sheet in the optimum position relative to the cut 230 position. Such a drum would allow for maximum speed of deposition of disposal bags 245. Alternatively, suction, grabbing, or other means may be used to place a cut bag 245 into place over the plastic sheet 210 during the production of disposable diapers.

### Third Embodiment

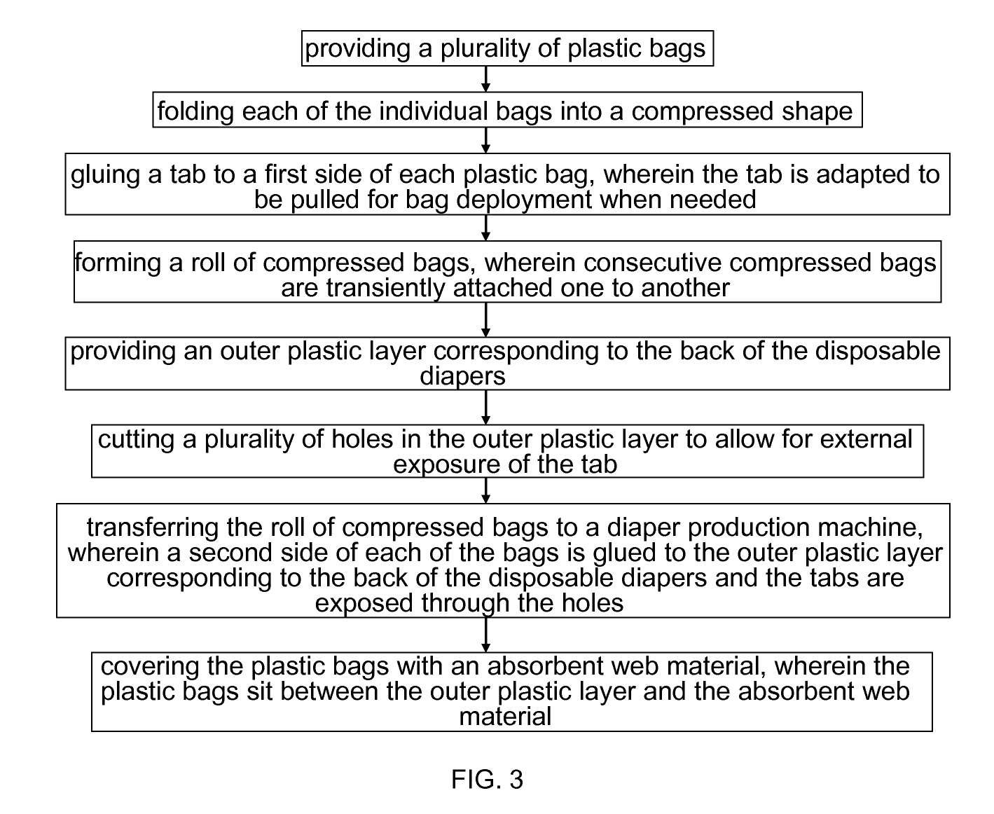

Attention is turned to FIG. 3, which shows a method associated with an embodiment of the instant invention. The figure details a method for positioning disposable bags during production of a plurality of disposable diapers, including the following: providing a plurality of plastic bags; folding each of the individual bags into a compressed shape; gluing a tab to a first side of each plastic bag, wherein the tab is adapted to be pulled for bag deployment when needed; forming a roll of compressed bags, wherein consecutive compressed bags are transiently attached one to another; providing an outer plastic layer corresponding to the back of the disposable diapers; cutting a plurality of holes in the outer plastic layer to allow for external exposure of the tab; transferring the roll of compressed bags to a diaper production machine, wherein a second side of each of the bags is glued to the outer plastic layer corresponding to the back of the disposable diapers and the tabs are exposed through the holes; and, covering the plastic bags with an absorbent web material, wherein the plastic bags sit between the outer plastic layer and the absorbent web material.

In one aspect of the method, there is a further step of completing production of the disposable diapers, wherein the plastic bags rest in a back portion of the disposable diapers and is adapted to be opened for receiving the diapers when soiled. Completion of diaper production may involve a plurality of steps including but not limited to adding additional layers, sewing, adding closure tabs, cutting individual diapers, organizing and packing of groups of disposable diapers. In another aspect of the method, the compressed bags in the roll are transiently attached directly or indirectly one to another in the roll. In another aspect of the method, the diaper production machine is adapted to produce hundreds of diapers or more per minute. In another aspect of the method, the tabs are made from plastic, paper, or a polymer material. In another aspect of the method, the step of transferring occurs in proximity to the diaper production machine. In another aspect of the method, the step of completing includes forming the disposable diapers, cutting individual diapers, arranging the disposable diapers in groups of diapers and packaging the groups of diapers. In another aspect of the method, the tab is adapted to be pulled without a user soiling his or her hands.

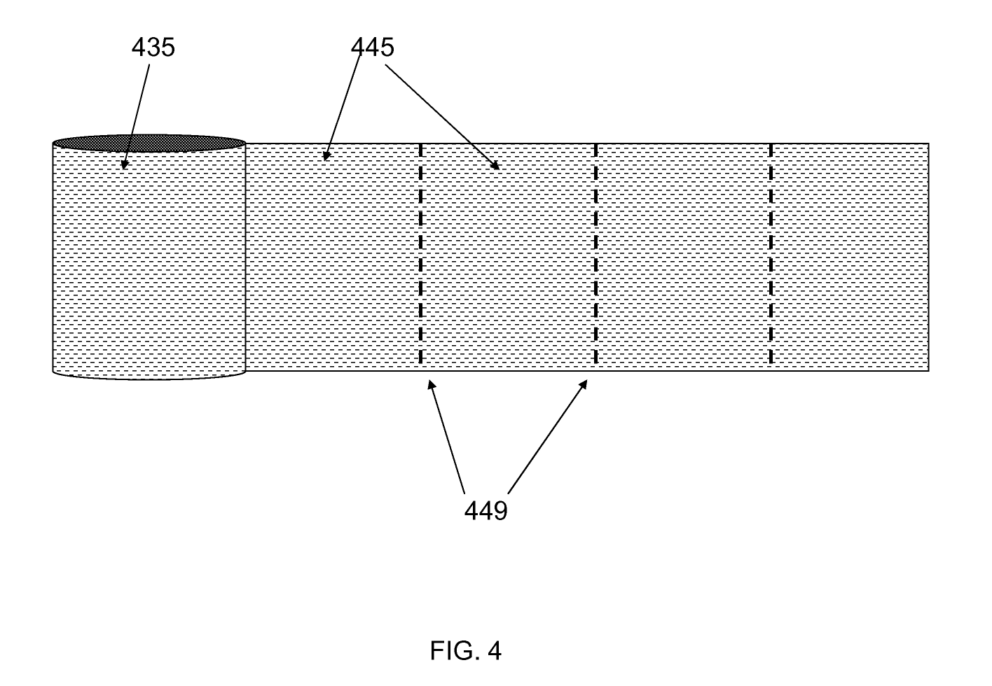

Attention is turned to FIG. 4 which shows a roll 435 of folded plastic disposal bags 445 for use in diaper (not shown) insertion. The folded plastic bags 445 are loosely connected through easily-cut connection zones 449 placed between individual plastic bags 445. The connection zones 449 are cut or otherwise treated so as to allow for liberation of individual bags 445 immediately prior to their insertion into the diaper production process. Cutting of a connection zone 449 exposes the open end of a plastic disposal bag 445 into which a soiled diaper (not shown) may be placed.

It is understood that a plastic bag 445 after receiving a soiled diaper to which said plastic bag 445 is attached may be closed by forming a knot or by using a tying element that may be provided with the bag 445 and/or diaper.

### Example

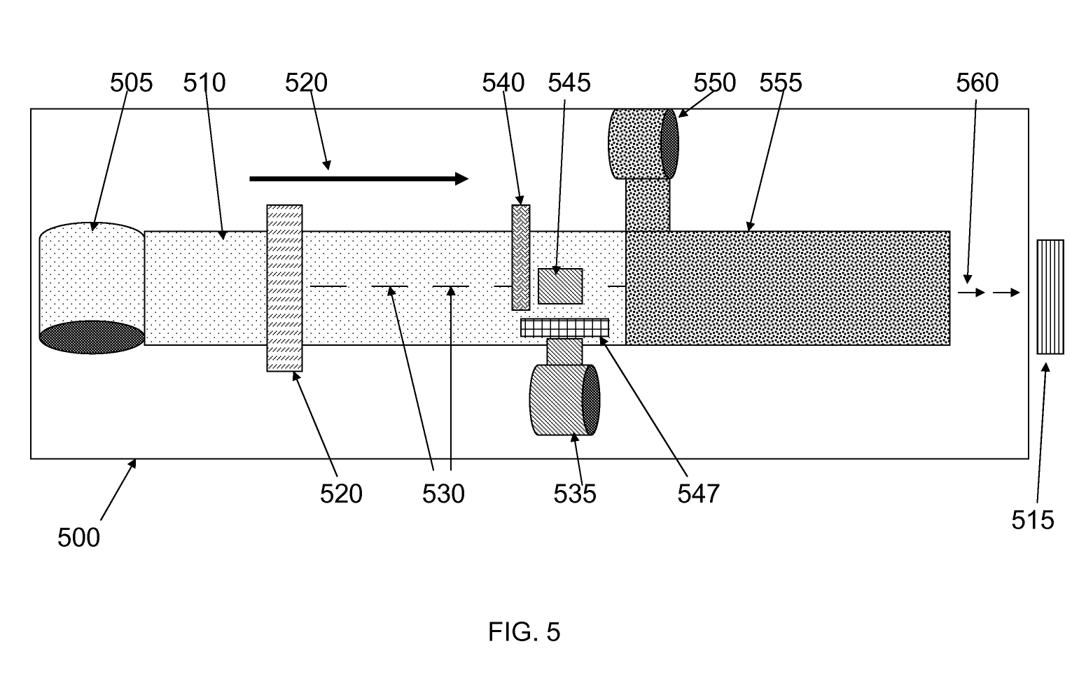

Attention is turned to FIG. 5 which shows a schematic drawing related to the present Example. A modified Joa diaper production machine 500 includes a roll 505 of nylon nonwoven fabric 510 that serves as the outer surface for a finished diaper 515. The fabric 510 is directed 520 at speeds of 350 meters per minute towards a cutting element 525 that places cuts 530 in said fabric510 in the same direction 520 of motion of said fabric 510. The cutting element 525 places a new cut 530 at a predetermined spacing along the nylon fabric 510. A gluing element 540 applies hot melt adhesive on the nylon fabric 510 to the left of each cut 530. A roll 535 of plastic disposal bags 545 is provided as is a cutting unit 547 which laser cuts off individual plastic bags 545 which are then glued to the nylon fabric in the general proximity of a cut 530. The cutting unit 547 may also include elements to press the cut disposal bag 545 in place. The cutting unit may alternatively be in the form of a roller that receives uncut bags 545 from the roll 535 and then cuts and pastes disposal bags 545 in place on the plastic sheet 510. A roll 550 of absorbent pad material 555 is provided and the pad material 555 is placed over the fabric 510 and its associated glued plastic disposal bags 545 and cuts 530 (no longer seen under the absorbent web material 255). Additional equipment and materials 560 are employed according to manufacturer's specifications in order to finish preparation of individual finished diapers 515. A typical roll 505, 535 & 555 may include thousands of meters of the appropriate material used in diaper preparation. It is understood that additional layers and materials including but not limited to elastic threads, hot melt adhesives, strips of tape or other closures, and inks used for printing decorations may be included in diaper production and are not shown for the purposes of clarity. As shown in the instant embodiment, the disposable plastic bag 545 is positioned on the inside of the nylon fabric 510 that forms the outer most layer of a finished diaper 515.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the, method or structure may include additional ingredients, steps and/or parts, but only if the additional ingredients, steps and/or parts do not materially alter the basic and novel characteristics of the claimed composition, method or structure.

It is understood that the instant invention may be fully or partially integrated into diaper production machines. The instant invention may be practiced on any machine or plurality of machines used in the production of diapers, tampons, or the like. The instant invention may be practiced to leave a portion of the diaper—in lieu of a separate tab—exposed from the cut in the rear of the diaper.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. The present invention could be employed for a wide variety of disposable products in which the soiled versions of said products require sanitary disposal. It is understood that the instant invention may be practiced with plastic bags “as are” without folding or compression; the bags would simply take up more space within the diaper but would be amenable to use according to embodiments of the instant invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is understood that the instant invention may be practiced with attachment of a plastic disposal bag on the outside of the diaper as well. A bag may be placed in a larger bag that is in turn glued to or otherwise attached to an outer surface of a diaper. In such an arrangement, there would be no requirement to cut a hole for a tab or otherwise place absorbent web material over the disposal bag, as the bag would be cut and glued to the outside, generally back, surface of the diaper. The bag would be either glued alone or placed inside a larger bag glued to an outer surface of a diaper. Such an embodiment might include a device for inserting a plastic disposal bag during disposable diaper production, including: a diaper production machine adapted to produce hundreds or more disposable diapers per minute; a roll of compressed plastic bags, wherein consecutive compressed plastic bags are connected one to another; a cutting unit adapted to cut the plastic bags into individual bags for delivery to the diaper production machine; a roll of plastic sheet adapted to be fed through the diaper production machine; and, a gluing element adapted to glue the individual bags onto an outer side of the plastic sheet.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for creating a disposable diaper with an internal bag for diaper disposal, including the following:
providing a plurality of connected plastic bags;
cutting said roll into individual bags;
folding each of said individual bags into a compressed shape;
gluing a tab to a first side of said plastic bag, wherein said tab is adapted to be pulled for bag deployment when needed;
forming a roll of compressed bags, wherein consecutive compressed bags are transiently attached one to another;
providing an outer plastic layer corresponding to the back of said disposable diaper;
cutting a hole in said outer plastic layer to allow for external exposure of said tab;
transferring said roll of compressed bag to a diaper production machine, wherein a second side of said bag is glued to said outer plastic layer corresponding to the back of said disposable diaper and said tab is exposed through said hole;
covering said plastic bag with an absorbent web material, wherein said plastic bag sits between said outer plastic layer and said absorbent web material; and,
completing production of said disposable diaper, wherein said plastic bag rests in a back portion of said disposable diaper and is adapted to be opened for receiving said diaper when soiled.

2. The method according to claim 1, wherein said step of forming is performed by placing each compressed bag into a pair of circular holders, wherein said holders are placed at the outer edges of each of said compressed bag and are glued with circular holders from a neighboring compressed bag to form a roll.

3. The method according to claim 1, wherein said step of forming is performed by placing said compressed bag into a larger holding bag having a hole to allow said tab to protrude, and wherein consecutive holding bags are glued together to form a roll.

4. The method according to claim 3, wherein said compressed bag is glued on a first side to an inner side of said larger holding bag.

5. The method according to claim 1, wherein said plastic bags are biodegradable.

6. The method according to claim 1, wherein said diaper is used for a toddler, child, medically unwell individual, or adult.

7. The method according to claim 1, wherein said diaper is realized as a woman's tampon.

8. The method according to claim 1, wherein said compressed shape has an exposed surface area of approximately 10% of that of the surface area of an uncompressed individual bag.

9. The method according to claim 1, wherein said gluing is performed by any of the following means: spray, direct application, contact application, heating, or by hand.

10. The method according to claim 1, wherein said outer plastic layer is realized as a long sheet of plastic adapted to be formed into an outer portion of said diaper.

11. The method according to claim 1, wherein said cutting is performed with a knife, laser, cutting tool, or razor.

12. The method according to claim 1, wherein said absorbent web material is adapted to absorb extruded bodily fluids.

13. The method according to claim 2, wherein said circular holders are made of plastic, paper, rubber, or polymer material.

14. A method for positioning disposable bags during production of a plurality of disposable diapers, including the following:
providing a plurality of plastic bags;
folding each of said individual bags into a compressed shape;
gluing a tab to a first side of each plastic bag, wherein said tab is adapted to be pulled for bag deployment when needed;
forming a roll of compressed bags, wherein consecutive compressed bags are transiently attached one to another;
providing an outer plastic layer corresponding to the back of said disposable diapers;
cutting a plurality of holes in said outer plastic layer to allow for external exposure of said tab;
transferring the roll of compressed bags to a diaper production machine, wherein a second side of each of said bags is glued to said outer plastic layer corresponding to the back of said disposable diapers and said tabs are exposed through said holes; and,
covering said plastic bags with an absorbent web material, wherein said plastic bags sit between said outer plastic layer and said absorbent web material.

15. The method according to claim 14, furthering including the step of completing production of said disposable diapers, wherein said plastic bags rest in a back portion of said disposable diapers and is adapted to be opened for receiving said diapers when soiled.

16. The method according to claim 14, wherein said compressed bags in said roll are transiently attached directly or indirectly one to another in said roll.

17. The method according to claim 14, wherein said diaper production machine is adapted to produce hundreds of diapers or more per minute.

18. The method according to claim 14, wherein said tabs are made from plastic, paper, or a polymer material.

19. The method according to claim 14, wherein said step of transferring occurs in proximity to said diaper production machine.

20. The method according to claim 14, wherein said step of completing includes forming said disposable diapers, cutting individual diapers, arranging said disposable diapers in groups of diapers and packaging said groups of diapers.

21. The method according to claim 14, wherein said tab is adapted to be pulled without a user soiling his or her hands.

22. A device for inserting a plastic disposal bag during disposable diaper production, including:
a diaper production machine adapted to produce hundreds or more disposable diapers per minute;
a roll of compressed plastic bags, wherein consecutive compressed plastic bags are connected one to another, and wherein each compressed plastic bag has associated with it a pull tab glued to a first side of said compressed plastic bag;
a cutting unit adapted to cut said plastic bags into individual bags for delivery to said diaper production machine
a roll of plastic sheet adapted to be fed through said diaper production machine;
a cutting element adapted to form regularly-spaced cuts in said plastic sheet;
a gluing element adapted to glue said individual bags onto said plastic sheet in general proximity to said cuts, wherein said tabs are exposed externally through said cuts in said plastic sheet; and,
a roll of absorbent web material adapted to be placed over said compressed plastic bags associated with said plastic sheet.

23. The device according to claim 22, further including a plurality of tabs, wherein said tabs are adapted to be attached to said individual plastic bags and allow for deployment of said bags.

24. The device according to claim 22, wherein said compressed plastic bags are connected one to another through the agency of a larger holding bag.

25. The device according to claim 22, wherein said cutting tool is associated with said diaper production machine.

26. The device according to claim 22, wherein said glue application element includes a spray component.

27. The device according to claim 22, wherein said diaper production machine is selected from one of the following vendors: Joa, Zuiko, or Fameccanica.
